(19) United States
(12) Patent Application Publication
(10) Pub. No.: US 2022/0262467 A1
(43) Pub. Date: Aug. 18, 2022

(54) HEALTH DATA PROCESSING SYSTEM AND HEALTH DATA PROCESSING METHOD

(71) Applicants: National University Corporation Hokkaido University, Sapporo-shi (JP); Hitachi, Ltd., Tokyo (JP)

(72) Inventors: Takahiro NAKAMURA, Tokyo (JP); Jyunichiro WATANABE, Tokyo (JP); Takashi TAKEMOTO, Tokyo (JP); Akiko TAMAKOSHI, Sapporo-shi (JP); Takashi KIMURA, Sapporo-shi (JP)

(21) Appl. No.: 17/575,855

(22) Filed: Jan. 14, 2022

(30) Foreign Application Priority Data
Feb. 12, 2021 (JP) 2021-020661

(51) Int. Cl. G16H 10/60; G06F 16/25

(52) U.S. Cl. CPC G16H 10/60; G06F 16/25

(57) ABSTRACT

A health data processing system in which an arithmetic unit can gain access to a database storing basic data of residents, health data of residents, and social capital index measurement data is provided. The health data processing system includes: an individual health condition analysis part which analyzes an individual health condition being each resident's health condition with use of the health data; a regional health condition analysis part which analyzes a regional health condition being a health condition of residents who belong to a region with use of the health data; an SCI derivation part which derives a regional SCI being a social capital index for every region with use of the social capital index measurement data; and an individually providing information generation part which generates information contributing to residents' health with use of the individual health condition, the regional health condition, and the regional SCI.

### CLAIM OF PRIORITY

The present application claims priority from Japanese patent application JP 2021-20661 filed on Feb. 12, 2021, the content of which is hereby incorporated by reference into this application.

### BACKGROUND

The present invention relates to a health data processing system.

As a background art of the present technical field, there is a prior art as follows. Patent Document 1 (Japanese Unexamined Patent Application Publication No. 2019-87239) discloses a regional comprehensive care business system including a database which has elderly person base data, care-required elderly-care insurance data, medical insurance data, and regional policy data. In the system, a regional management support function part outputs a quantitative analysis report about elderly person information or a service usage status by an undertaking unit from the data on the medical treatment and the care, and a qualitative report based on an index representing a status change in mind and body status item; a regional information management function unit outputs an activity evaluation result pertaining to a quantity or quality of service in an individual form method by the undertaking unit; and an elderly person information management function unifies base information on an elderly person, a mind and body status usage service situation, and a medical treatment situation, which supports effect verification of a care plan by reference to a past history about these actions and a review policy consideration of a next-term plan (see Abstract).

Further, a significant correlation between a social capital index and health consciousness is disclosed in Non-patent Document 1 (Masashige Saito, et.al., “Development of an instrument for community-level health related social capital among Japanese older people: The JAGES Project”, Journal of Epidemiology 27 (2017) 221e227, Feb. 4, 2017).

### SUMMARY

For example, due to the pandemic of Covid19, social properties such as people's chance of interchange is degraded, and a situation has occurred where regional division tends to be produced. As in the regional comprehensive care business system disclosed in Patent Document 1 (Japanese Unexamined Patent Application Publication No. 2019-87239), the conventional health service can analyze individual health conditions considering the health property of the region. However, social properties including the strength of relationship of people in the region is not considered.

On the other hand, as shown in Non-patent Document 1 (Masashige Saito, et.al., “Development of an instrument for community-level health related social capital among Japanese older people: The JAGES Project”, Journal of Epidemiology 27 (2017) 221e227, Feb. 4, 2017), there is a social capital index (SCI) as an index which shows the strength of the relationship of people, such as “reliance”, “social norm”, and a “social network,” and a constant correlation exists between SCI and health consciousness.

In the “with corona age” and the “After corona age,” it is feared that health properties are unfavorably affected by the fall in the regional SCI. Thus, when regional SCIs are different, it is assumed that required policies and contents of a care differ from region to region. However, in the conventional technology, those are not simultaneously measured and analyzed and reflected in health improvement policies.

It is an object of the present invention is to realize a mechanism of promoting health by a policy and advice through measurement and analysis of health properties and social properties.

A typical example of the present invention shown in the present application is as follows: A health data processing system includes a computer having an arithmetic unit which performs predetermined processing and a memory device connected to the arithmetic unit. The arithmetic unit can gain access to a database which stores basic data of residents, health data of residents, and social capital index measurement data. The health data processing system further includes: an individual health condition analysis part in which the arithmetic unit analyzes an individual health condition being each resident's health condition with use of the health data; a regional health condition analysis part in which the arithmetic unit analyzes a regional health condition being health condition of residents who belong to the region with use of the health data; an SCI derivation part in which the arithmetic unit derives a regional SCI being a social capital index for every region with use of the social capital index measurement data; and an individually providing information generation part in which the arithmetic unit generates information contributing to the health of the residents with use of the individual health condition, the regional health condition, and the regional SCI.

According to one mode of the present invention, information suitable for each resident can be provided. The problem, configuration, and effects other than the above described will be made clear by explanation of the embodiment below.

### BRIEF DESCRIPTION OF THE DRAWINGS

FIG. 1 is a diagram showing a configuration of a regional health data processing system according to the present embodiment;

FIG. 2 is a diagram showing detailed configurations of a health service effect analysis/service ranking generation part and an individually providing information generation part;

FIG. 3 is a diagram showing a detailed configuration of an individual SCI problem analysis part;

FIG. 4 is a diagram showing an example of the operation by a sociality reference value arithmetic unit; and

FIG. 5 is a diagram showing an example of the operation by an individual social arithmetic unit.

### DETAILED DESCRIPTION

Hereinafter, with reference to the drawings, embodiments of the present invention will be described.

In the following embodiments, when it is necessity for the sake of convenience, the description will be divided into a plurality of sections or embodiments. However, unless otherwise specified, they are not irrelevant to each other. There are some or all of the modifications, details, supplementary explanations, and the like. Further, in the following embodiments, when referring to the number of elements (including the number, numerical value, quantity, range, etc.), unless especially clearly indicated and clearly limited to a specific number in principle, etc., it is not limited to the specific number, and may be more or less than the specific number.

Further, needless to say, in the following embodiments, the constituent elements (including element steps and the like) are not necessarily indispensable unless otherwise specified and apparently essential in principle. Similarly, in the following embodiments, when referring to the shape, positional relationship, etc., of components, etc., unless otherwise specified, and in principle, it is considered that this is not clearly the case, it includes those that are substantially approximate or similar to the shape etc. The same applies to the above numerical values and ranges.

Hereinafter, embodiments of the present invention will be described in detail based on the drawings. In all the drawings for explaining the embodiments, the same members are denoted by the same reference symbols in principle, and the repeated explanation thereof is omitted.

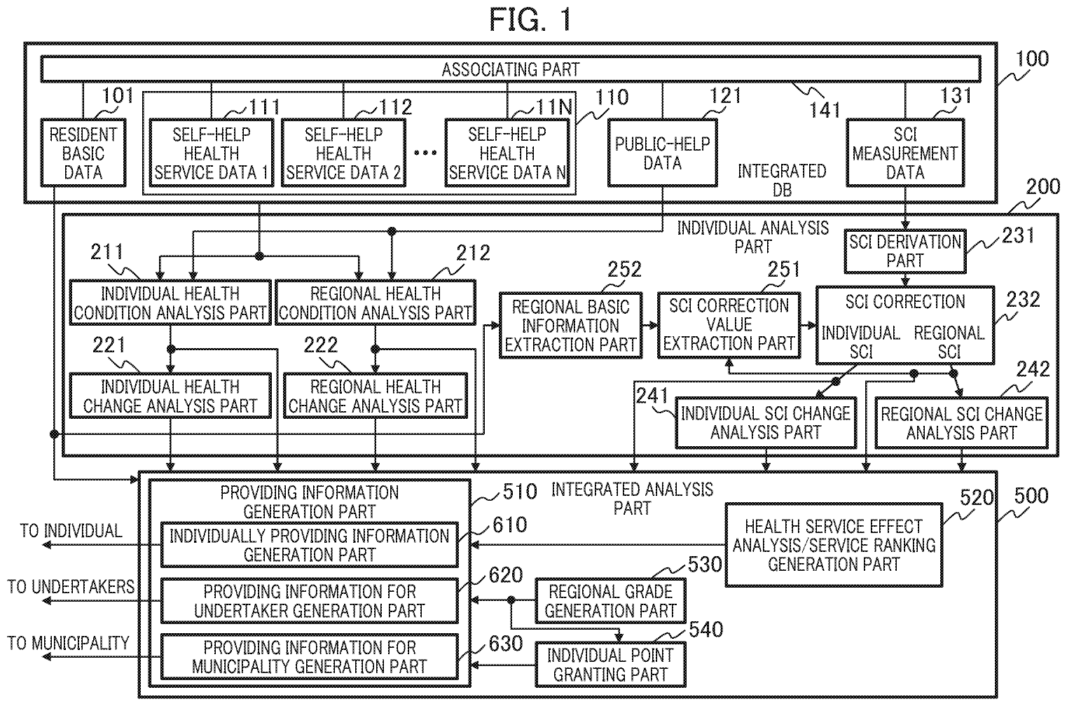

FIG. 1 is a diagram showing a configuration of a regional health data processing system according to the embodiment of the present invention.

The regional health data processing system of the embodiment of the present invention analyzes an individual health condition, the health property of the region, and the social capital index (regional SCI) of the region collectively, and proposes health advice to an individual considering the regional property.

The regional health data processing system of the present embodiment includes an integrated database 100, an individual analysis part 200, and an integrated analysis part 500. The integrated database 100 has the resident basic data 101, the self-help health service data 110, the public-help data 121, and the data 131 for SCI measurement. The resident basic data 101 contains a residents' address, age, sex, etc. The self-help health service data 110 includes self-help health service data 1 to N (111 to 11N) which contains participation information of various self-help health services and health data (general health data such as height, weight, etc., special health data such as stools, mother's milk, urine, etc.). The public-help data 121 contains medical checkup data, a medical statement, a care medical statement, etc. The data 131 for SCI measurement is data for measuring a social capital index (SCI) such as a result of a predetermined questionnaire, sensing data, etc. Questionnaires relate to, for example, participation in an event, activity in the region, etc., which makes it possible to know the relationship between SCI and the health questionnaire. The sensing data is sensing data of the voice, the camera, the amount of vibration, etc. in a regional event etc., for example, and a degree of activity etc. can be measured. The integrated database 100 is formed in which the correlation part 141 associates various data using a resident ID uniquely given to residents.

The individual analysis part 200 includes: an individual health condition analysis part 211; a regional health condition analysis part 212; an individual health change analysis part 221; a local health change analysis part 222; a regional basic information extraction part 252; an SCI correction value extraction part 251; an SCI derivation part 231; an SCI correction part 232; an individual SCI change analysis part 241; and a regional SCI change analysis part 242.

The individual health condition analysis part 211 analyzes individual health condition and health risk from the self-help health service data 110 and the public-help data 121. The regional health condition analysis part 212 analyzes a statistical health condition and the health risk of residents of the region. The individual health change analysis part 221 analyzes change in the individual health condition. The regional health change analysis part 222 analyzes change in the health condition of the residents of the region. The regional basic information extraction part 252 extracts regional basic information, such as population density and age distribution of the region. The SCI correction value extraction part 251 outputs an SCI correction value from extracted SCI and regional basic information. It is known that there is a relationship between health consciousness and SCI. By correcting SCI using regional basic information, the deviation for every area is eliminated and a suitable SCI which do not depend on the region can be outputted.

The SCI derivation part 231 extracts an individual SCI from the data 131 for SCI measurement, and derives a regional SCI from the derived individual SCI. The SCI correction part 232 corrects the derived individual SCI and regional SCI for every region. The individual SCI change analysis part 241 analyzes change in the individual SCI. The regional SCI change analysis part 242 analyzes change in the regional SCI.

The integrated analysis part 500 includes: a providing information generation part 510; a health service effect analysis/service ranking generation part 520; a regional grade generation part 530 which derives a regional grade by comprehensively judging from each index; and an individual point grant part 540. The providing information generation part 510 includes: an individually providing information generation part 610; a providing information for undertaker generation part 620; and a providing information for municipality generation part 630.

The health service undertakers to whom the regional health data processing system of the present embodiment provide information by the providing information for undertaker generation part 620 are undertakers who require analysis and evaluation of human health and are medical undertakers, nursing care service providers, medical analysis organization (for example, a gene-analysis organization, a blood test organization, etc.), medicine manufacturers, and health food manufacturers.

The SCI includes a plurality of indexes. Those indexes correspond to, for example, social cohesion (reliance on people, attachment to the region), reciprocity (participation in an event or a municipality's event), and civic participation (number of family members, relatives, and friends that can be met or consulted with). Further, a difference within the region of social deprivation index being an index about poverty or an economic index such as tax-payment data held by the municipality may be used as SCI. A prior research implies that when an intraregional disparity within the region is large, the degree of satisfaction is low. Therefore, it becomes possible to extract a problem better suited to an actual situation and generate advice by using an economic index being different in terms of viewpoint from the SCI described above.

There is a close correlation between the regional SCI and regional basic information such as population density and age distribution of the region. Therefore, it may be possible to analyze the regional SCI as an index including effects of regional basic information. However, by finding the deviation from the regional SCI predicted based on the regional basic information by the regional basic information extraction part 252 and the SCI correction value extraction part 251, it becomes possible to use, as an index, the regional SCI peculiar to the region from which effects of the regional basic information is eliminated.

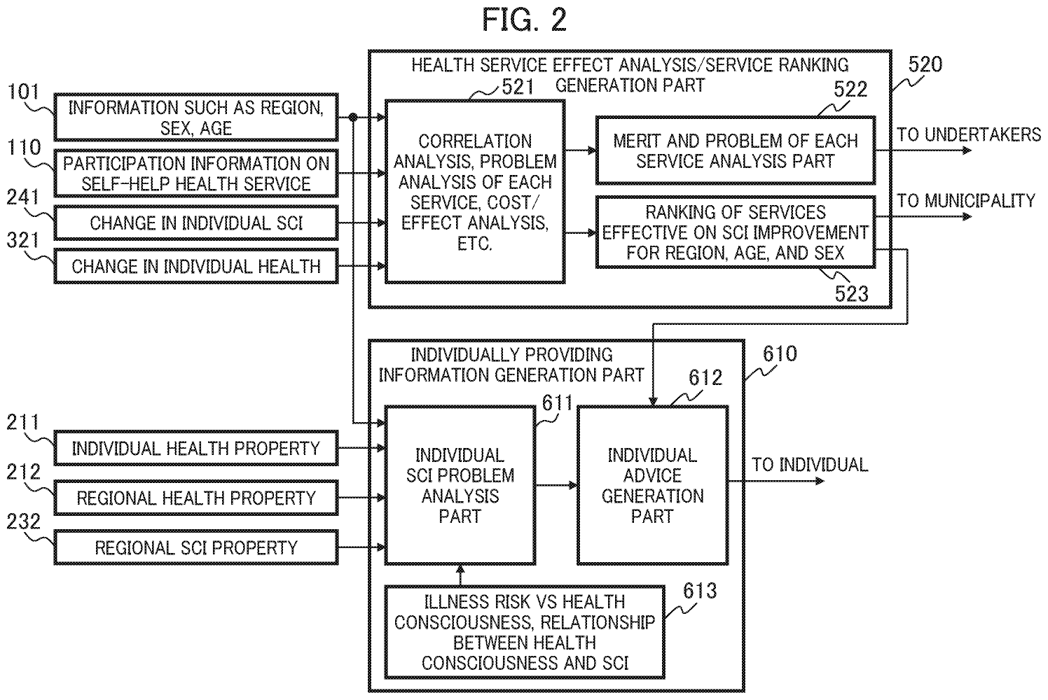

FIG. 2 is a diagram showing detailed configurations of a health service effect analysis/service ranking generation part 520 and an individually providing information generation part 610.

The health service effect analysis/service ranking generation part 520 analyzes the degree of contribution of each service to the regional SCI, analyzes the degree of contribution of each service to the individual health improvement and improvement in health consciousness. It further analyzes the degree of contribution to various indexes (regional SCI, individual health condition, individual health consciousness, and the regional health condition) for every region, and generates the ranking of self-help health services. Specifically, with use of information such as region, sex, age, etc., of people to be analyzed included in the resident basic data 101, participation information on various services included in the self-help health service data 110, the individual SCI change information outputted from the individual SCI change analysis part 241, and change information on the individual health condition outputted from the individual health change analysis part 221, the health service effect analysis/service ranking generation part 520 analyzes the effects of various health services on the health condition and the SCI and quantitatively derives effects on each item of the health property and SCI for every region, age, and sex. Further, for region, age, and sex, according to effects on the health property and SCI, the health service effect analysis/service ranking generation part 520 generates ranking of self-help health services. Still further, the health service effect analysis/service ranking generation part 520 analyzes merits and problems of each self-help health service and provides effects on health property and each item of SCI and ranking to the health service undertakers, who can utilize them as indexes for them to achieve improvement in health services. Moreover, it becomes possible to examine regional SCI improvement policy for every region by providing, to a municipality, information in which ranking, for every region, age, and sex, of the services effective in improving each SCI is listed, and precision and efficiency of the examination can be improved.

The individually providing information generation part 610 includes an individual SCI problem analysis part 611 and an individual advice generation part 612. With use of information such as region, sex, age, etc., of people to be analyzed included the resident basic data 101, individual health property information to be outputted from the individual health condition analysis part 211 and the individual health change analysis part 221, information about regional health property to be outputted from the regional health condition analysis part 212 and the regional health change analysis part 222, the regional SCI property outputted from the SCI correction part 232, and knowledge-base information (for example, a relation between illness risk and health consciousness, a correlation between health consciousness and SCI, etc.) 613, the individual SCI problem analysis part 611 analyzes an SCI problem with respect to each individual. The individual advice generation part 612 generates advice for an individual by using the problem derived by the individual SCI problem analysis part 611 and the health service ranking effective in SCI improvement derived by the health service effect analysis/service ranking generation part 520.

The individual SCI problem analysis part 611 analyzes individual and regional health risk together with the regional SCI collectively, and derives an SCI that an individual should raise. By improvement in the SCI, both the health and the SCI can be improved. Moreover, the speed of effect verification of the health service can be improved by using SCI with shorter time until an effect appears compared with health condition as part of the index. Moreover, it becomes possible to discover at an early stage a problem related to a social property such as regional division, which can contribute to maintenance and improvement of the regional social property. Furthermore, by using the regional SCI excluding effects such as population density and age distribution (aging rate) which are hard to intervene as an index, a problem whose intervening effects are high can be extracted.

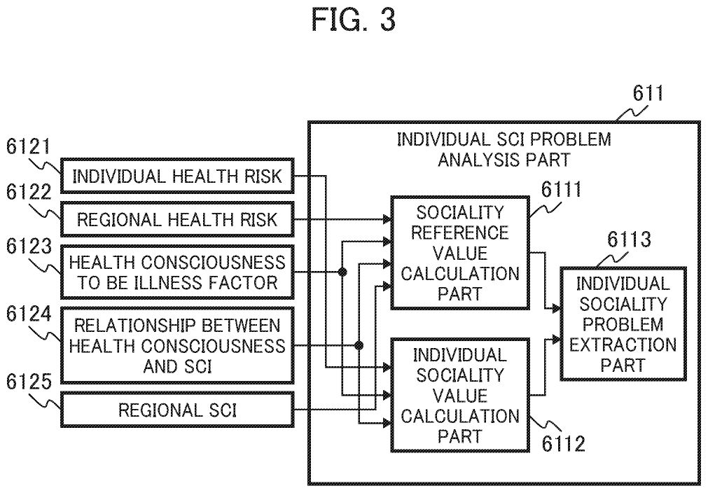

FIG. 3 is a diagram showing the detailed configuration of an individual SCI problem analysis part 611.

The individual SCI problem analysis part 611 includes a sociality reference value calculation part 6111, an individual sociality calculation part 6112, and an individual sociality problem extraction part 6113.

The sociality reference value calculation part 6111 derives a sociality reference value Scref used as a reference of an individual SCI problem from a regional health risk 6122 peculiar to the region in which a regional health condition outputted from the regional health condition analysis part 212 is arranged in terms of the risk, health consciousness 6123 to be a cause of illness in each health risk obtained in analysis of results of the knowledge base and the questionnaire, a relationship 6124 between the health consciousness obtained by analysis of the result of knowledge base or the questionnaire and SCI, and the regional SCI 6125. The individual sociality calculation part 6112 derives a conversion value Si of each SCI to an individual health risk from an individual health risk 6121 in which the individual health condition outputted from the individual health condition analysis part 211 are arranged in terms of the risk, the health consciousness 6123 to be a cause of illness in each health risk obtained by analysis of the knowledge base or a result of the questionnaire, and the relationship 6124 between the health consciousness obtained by the knowledge base and analysis of the result of the questionnaire and the SCI. The health consciousness 6123 to be the cause of illness and the relationship 6124 between the health consciousness and the SCI are contained in the resident basic data 101.

The individual sociality problem extraction part 6113 analyzes the relationship between the conversion value Si of each SCI and the sociality reference value Scref. When the conversion value Si of each SCI is greater than the sociality reference value Scref, it is judged that the SCI concerned is an SCI which an individual should raise. The sociality reference value Scref to be a reference includes a health risk peculiar to the region and the regional SCI. The SCI judged in the individual sociality problem extraction part 6113 is to be an SCI problem derived in consideration of the regional health property and the regional SCI. The individual sociality problem extraction part 6113 may sort and output the SCI problems in the order of the sociality reference value Scref.

Thus, by deriving the individual SCI problem, it becomes possible to clarify an individual problem for improving not only an individual health property but also a regional health property and a regional SCI. Further, by providing policy information and advice for improving the derived SCI problem, it becomes possible to improve not only the individual health property but also the regional health property and the regional SCI. Moreover, by using the regional SCI except effects of population density, age distribution (aging rate), etc. being hard to intervene as an index, a problem with high intervening effect can be extracted.

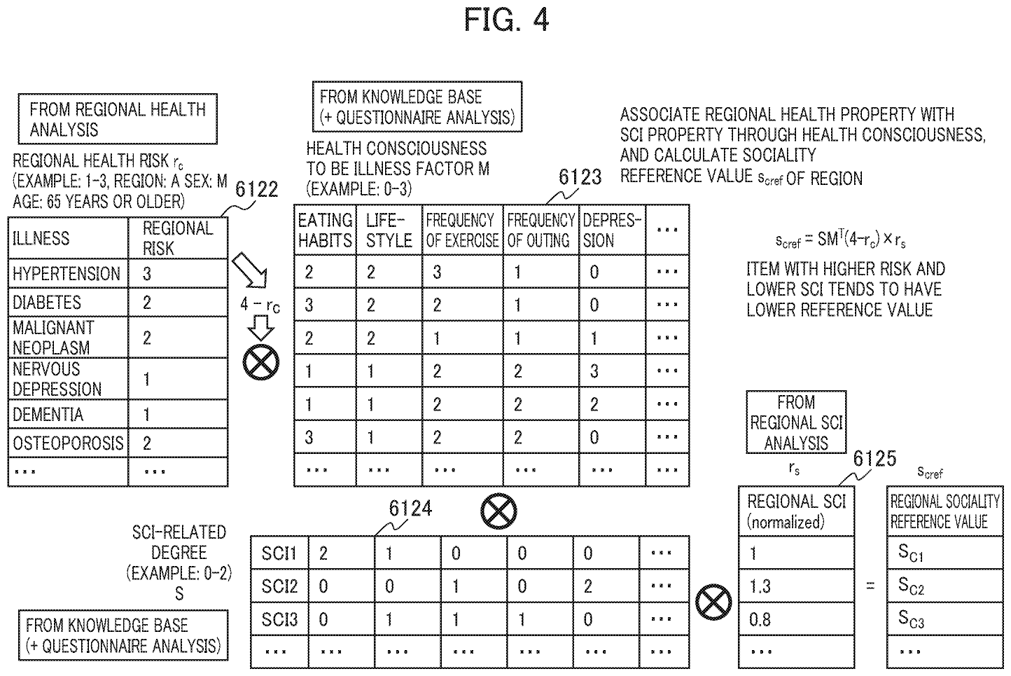

FIG. 4 is a diagram showing an example of the operation by the sociality reference value calculation part 6111.

For example, the regional health risk rc (6122) is graded about each illness in three steps. The health consciousness M to be a cause of illness (6123) is graded about the health consciousness related to each illness in, for example, four steps. The SCI degree of relationship S (6124) which shows the relationship between the health consciousness and SCI is graded about the relationship between the health consciousness and the SCI in, for example, three steps. For the regional SCI rs (6125), the numerical value standardized, for example, on the basis of the average value is used. For the calculation of the sociality reference value Scref, it may serve the purpose to reverse a sign and to use 4-rc so that when a regional health risk is higher, a numerical value may become lower. These are multiplied as illustrated, and the sociality reference value Scref is derived. The derived sociality reference value Scref becomes lower as the health risk of the region is higher, and becomes lower as the regional SCI is lower.

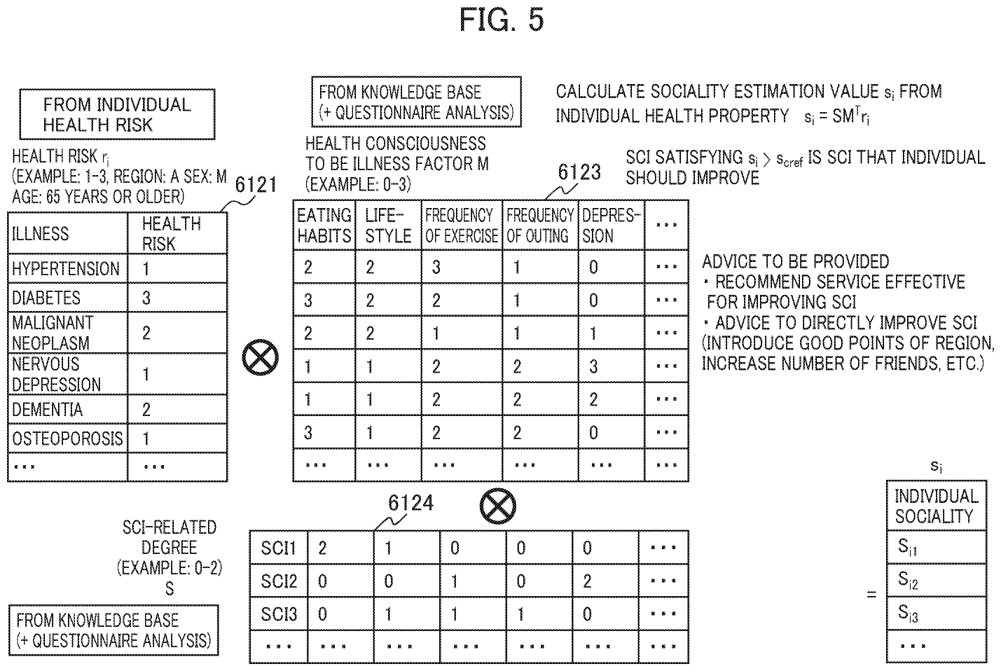

FIG. 5 is a diagram showing an example of the operation by the individual sociality calculation part 6112.

For example, the individual health risk ri (6121) is graded about each illness in three steps. The case of the health consciousness M leading to the illness (6123) and the SCI degree of association S (6124) are the same as the operation by the sociality reference value calculation part 6111 shown in FIG. 4. These are multiplied as illustrated, and the individual sociality estimation value Si is derived. When the derived individual sociality Si is higher than the sociality reference value Scref, it is judged the SCI concerned is the SCI problem that the individual should improve. As described above, the sociality reference value Scref to be a reference of SCI problem judgment becomes lower as the health risk of the region is higher, and becomes lower as regional SCI is lower. Therefore, the SCI problem in consideration of the health risk of the region or a regional SCI can be judged.

As explained above, the regional health data processing system of the present embodiment includes: the individual health condition analysis part 211 which analyzes the individual health condition being each resident's health condition using residents' health data (self-help health service data 110); the regional health condition analysis part 212 which analyzes the regional health condition which is the health condition of the residents belonging to the region using residents' health data 110; the SCI derivation part 231 which derives the regional SCI being a social capital index for every region with use of the social capital index measurement data (SCI measurement data 131); and the individually providing information generation part 610 which generates information contributing to residents' health with use of the individual health condition, regional health condition, and the regional SCI. Therefore, the residents can enjoy policy information and advice in consideration of individual health condition, health property of residing region, and area SCI, and information more suitable for each resident can be provided.

Moreover, the system has the SCI correction part 232 which corrects the regional SCI with use of regional basic information including population density and age distribution of the region. By finding the deviation from the regional SCI predicted based on the regional basic information, a suitable SCI can be calculated, using the area SCI peculiar to the region except the effect of the regional basic information as an index.

Moreover, the SCI derivation part 231 derives an individual SCI which is a social capital index of each resident with use of the social capital index measurement data 131. Further, the regional health data processing system includes: an individual health change analysis part 221 which analyzes change in the individual health condition; an individual SCI change analysis part 241 which analyzes change in the derived individual SCI; and a health service effect analysis part (health service effect analysis/service ranking generation part 520) which calculates effects on a regional health condition by the health service with use of the resident basic data 101, change in the individual health condition, change in individual SCI, and participation information of the health service. Therefore, both the health and the SCI can be improved. Moreover, the speed of effect verification of health service can be improved by using SCI with shorter time until an effect appears compared with health condition for a part of index. Moreover, problems related to social properties, such as regional division can be discovered at an early stage, which can contribute to the maintenance and improvement of the regional social properties. Further, a problem with high intervening effect can be extracted.

Moreover, using social capital index measurement data 131, the SCI derivation part 231 derives the individual SCI which is each resident's social capital index. Further, the individually providing information generation part 610 includes: a sociality reference value calculation part 6111 which calculates the sociality reference value to be a reference of the individual SCI with use of a regional health condition, information on a relationship between a health condition and health consciousness, information on a relationship between the health consciousness and the individual SCI, and the regional SCI; a sociality calculation part 6112 which calculates individual sociality with use of an individual health condition, information on a relationship between a health condition and the health consciousness, and information on a relationship between the health consciousness and the individual SCI; a sociality problem extraction part 6113 which extracts an individual SCI problem according to a relationship between the individual sociality and the sociality reference value. Therefore, not only an individual health property but also the regional health property and the regional SCI can be improved. Moreover, the problem of high intervention effect can be extracted.

In addition, the present invention is not limited to the above embodiment, and includes various modifications and applications within the scope of the invention described above in the claims. For example, the embodiment described above have been described in detail for easy understanding of the present invention, and the present invention is not necessarily limited to those having all the configurations described. Also, a part of a configuration of one embodiment can be replaced with a configuration of another embodiment. Also, a configuration in one embodiment can be added to a configuration in another embodiment. Still further, with respect to a part of a configuration in each embodiment, addition, deletion, and replacements of the other configurations may be made.

Further, part or all of each of the configurations, functions, processing parts, processing means, etc. described above may be realize through hardware by designing them, for example, with use of integrated circuits. Alternatively, they may be realized through software with use of a processor interpreting a program for realizing each function and carrying it out.

Information such as a program realizing each function, a table, and a file can be included in a recording device such as a memory, a hard disk, or a solid state drive (SSD), or in a recording medium such as an IC card, an SD card, and a DVD.

In addition, it is considered that a control line or a information line is necessary for description, and both of the control line and the information line are not necessarily shown for a product. In practice, it may be considered that almost all configurations are connected to each other.

What is claimed is:

1. A health data processing system including: a computer having an arithmetic unit performing predetermined processing and a memory device connected to the arithmetic unit,
the arithmetic unit being able to gain access to a database storing basic data of residents, health data of residents, and social capital index measurement data,
the health data processing system including:
an individual health condition analysis part in which the arithmetic unit analyzes an individual health condition being each resident's health condition with use of the health data;
a regional health condition analysis part in which the arithmetic unit analyzes a regional health condition being health condition of residents who belong to the region with use of the health data;
an SCI derivation part in which the arithmetic unit derives a regional SCI being a social capital index for every region with use of the social capital index measurement data; and
an individually providing information generation part in which the arithmetic unit generates information contributing to the health of the residents with use of the individual health condition, the regional health condition, and the regional SCI.

2. The health data processing system according to claim 1,
wherein the arithmetic unit includes an SCI correction part which corrects a regional SCI with use of regional basic information including regional population density and age distribution.

3. The health data processing system according to claim 1,
wherein the health data of residents includes participation information of health service provided to residents,
wherein the SCI derivation part derives an individual SCI being a social capital index of each resident with use of the social capital index measurement data,
wherein the health processing system includes:
an individual health change analysis part in which the arithmetic unit analyzes change in the individual health condition;
an individual SCI change analysis part in which the arithmetic unit analyzes change in the derived individual SCI; and
a health service effect analysis part in which the arithmetic unit calculates effects on a regional health condition by the health service with use of the basic data, change in the individual health condition, change in the individual SCI, and participation information of the health service.

4. The regional heal data processing system according to claim 1,
wherein the SCI derivation part derives an individual SCI being each resident's social capital index with use of social capital index measurement data,
wherein the individually providing information generation part includes:
a sociality reference value calculation part in which the arithmetic unit calculates a sociality reference value being a reference of the individual SCI with use of the regional health condition, information on a correlation between a health condition and health consciousness, information on a correlation between the health consciousness and the individual SCI, and the regional SCI;
a sociality calculation part in which the arithmetic unit calculates individual sociality with use of the individual health condition, information on the correlation between the health condition and a health consciousness, and information on the correlation between the health consciousness and the individual SCI; and
a sociality problem extraction part in which the arithmetic unit extracts an individual SCI problem with use of the relation between the sociality reference value and the individual sociality.

5. A health data processing method performed by a health data processing system,
wherein the health data processing system includes: a computer having an arithmetic unit performing predetermined processing and a memory device connected to the arithmetic unit,
wherein the arithmetic unit can gain access to a database storing basic data of residents, health data of residents, and social capital index measurement data,
wherein the health data processing method includes the steps of:
an individual health condition analysis in which the arithmetic unit analyzes an individual health condition being each resident's health condition with use of the health data;
a regional health condition analysis in which the arithmetic unit analyzes a regional health condition being health condition of residents who belong to the region with use of the health data;
an SCI derivation in which the arithmetic unit drives a regional SCI being a social capital index for every region with use of the social capital index measurement data; and
an individually providing information generation in which the arithmetic unit generates information contributing to the health of the residents with use of the individual health condition, the regional health condition, and the regional SCI.

6. The health data processing method according to claim 5,
wherein the arithmetic unit includes an SCI correction part which corrects a regional SCI with use of regional basic information including regional population density and age distribution.

7. The regional health data processing method according to claim 5,
wherein the health data of residents includes participation information of health service provided to the residents,
wherein, in the SCI derivation step, the arithmetic unit derives an individual SCI being each resident's social capital index with use of the social capital index measurement data,
wherein the health data processing method includes the steps of:
an individual health change analysis in which the arithmetic unit analyzes change in the individual health condition;
an individual SCI change analysis in which the arithmetic unit analyzes change in the derived individual SCI; and
a health service effect analysis in which the arithmetic unit calculates effects on the regional health condition by the health service with use of the basic data, the change in the individual health condition, the change in the individual SCI, and participation information of the health service.

8. The regional health data processing method according to claim 5,
wherein, in the SCI derivation step, the arithmetic unit derives an individual SCI being each resident's social capital index with use of the social capital index measurement data,
wherein the individually providing information generation method includes the steps of:
a sociality reference value calculation in which the arithmetic unit calculates a sociality reference value being a reference of the individual SCI with use of the regional health condition, information on a correlation between a health condition and health consciousness, information on a correlation between health consciousness and the individual SCI, and the regional SCI;
a sociality calculation in which the arithmetic unit calculates individual sociality with use of the individual health condition, information on the correlation between the health condition and a health consciousness, and information on the correlation between the health consciousness and the individual SCI; and
a sociality problem extraction in which the arithmetic unit extracts an individual SCI problem with use of the relation between the sociality reference value and the individual sociality.
